(19) United States
(12) Patent Application Publication
(10) Pub. No.: US 2008/0114323 A1
(43) Pub. Date: May 15, 2008

(54) Article With Secondary Fastening System

(75) Inventors: Mark James Kline, Okeana, OH (US); Jeromy Thomas Raycheck, Lebanon, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/934,102

(22) Filed: Nov. 2, 2007

Related U.S. Application Data
(60) Provisional application No. 60/856,463, filed on Nov. 3, 2006.

(51) Int. Cl. A61F 13/15

(52) U.S. Cl. 604/385.03; 604/387; 604/389; 604/391

(57) ABSTRACT

Disposable absorbent articles having a primary fastening system and a secondary fastening system, wherein the primary fastening system comprises at least a first component and at least a second component and the first component interlocks with the second component; and the secondary fastening system is located on at least a portion of the primary fastening system; wherein the secondary fastening system comprises a secondary fastening member having a first surface and a protective portion configured to cover at least a portion of the first surface.

### CROSS REFERENCE TO RELATED APPLICATION

This application claims the benefit of U.S. Provisional Application No. 60/856,463, filed Nov. 3, 2006, the substance of which is incorporated herein by reference.

### FIELD OF THE INVENTION

The present invention relates to absorbent articles including but not limited to diapers, training pants, and incontinence pads. More particularly, the present invention relates to disposable absorbent articles having a secondary fastening system for fastening the article in a configuration for disposal.

### BACKGROUND OF THE INVENTION

Many different types of refastenable fastening devices are known, including ties, pins, hook-and-loop systems, hook-and-eye systems, buttons, snaps, interlocking shapes, buckles, adhesive tapes, cohesive surfaces, and zippers and other slide connectors. Such fasteners have been used on a variety of products, both durable and disposable. Typical uses include envelopes, clothing, diapers, packages, footwear, construction closures, general attachment needs and feminine hygiene products.

Primary and secondary fastening devices are of particular use for absorbent articles such as diapers. Primary fastening devices refer to fastening devices used to fasten a rear portion of the diaper to a front portion of the diaper for fitting the diaper to a wearer. Secondary fastening devices refer to fastening devices used to fasten the diaper in a configuration for disposal. For example, when removing a diaper, it is common for a caregiver to roll the diaper from front to back and fasten the diaper in the rolled configuration. Commonly, the primary fastening device may be used as a secondary fastening device.

Some fastening devices, such as hook-and-loop or adhesive tapes, require aligning an engaging surface with a landing surface. These may be referred to as surface fastening devices herein. While surface fastening devices can result in an effective closure, they often result in misapplication and/or poor alignment of the elements being connected. Further, hook-and-loop fasteners can become ineffective due to compression and contamination, or can harm surrounding materials. With an adhesive system, improperly fastening the device may render the entire product unusable. For example, in diaper applications, repositioning a tape tab that has been fastened improperly may result in tearing the outer cover of the diaper. Further, adhesive systems are prone to contamination-induced performance problems. For example, an adhesive tab that has contacted baby lotion or baby powder may have less ability to connect to its landing surface. In order to help prevent such problems, the use of these types of fasteners often requires inefficient designs and extra material usage, which can add to the cost of the products.

Articles including fastening devices comprising, for example, a primary fastening device such as a macrofastener (described more fully below) and a secondary fastening device such as an adhesive or hook fastener (of a hook-and-loop fastening system) positioned on the primary fastening device are known in the art. Typically, the macrofastener comprises any first and second non-planar interlocking structures, the first interlocking structure being provided on one of a front or rear of the article and the second interlocking structure being provided on the other of a front or rear of the article. Commonly the first interlocking structure may comprise a tab and the second interlocking structure may comprise a slot for receiving the tab. The adhesive or hook fastener is provided on the tab. With this configuration, the adhesive may be contaminated during insertion of the tab through the slot or during wearing thus rendering the adhesive ineffective for use during disposal. Similarly, the hook fastener (of the hook-and-loop fastening system) may become entangled with the slot as the tab is inserted through the slot, thus a hook fastener may interfere with convenient fastening of the primary fastener. However, configuring the tab and slot for disposal can be cumbersome for the user.

It would be advantageous to provide an improved fastening device. It would also be advantageous to provide a refastenable fastening device that allows adjustability and that is suitable for many uses, including disposable products, absorbent articles, and especially disposable absorbent articles. Further, it would be advantageous to provide an absorbent article having a fastening device that can easily be engaged in many different configurations, such as when the wearer is sitting, lying down, or standing. It would also be advantageous for the primary fastening system to include a secondary fastening system which did not interfere with convenient fastening of the primary fastener or become contaminated prior to disposal, yet was configured to enable convenient disposal of a used product.

### SUMMARY OF THE INVENTION

Disposable absorbent articles having a primary fastening system and a secondary fastening system for fastening the article in a configuration for disposal are provided. Further, secondary fastening systems suitable for use with disposable absorbent articles and interlocking primary fastening systems are provided. Further, secondary fastening systems with protected portions are provided.

In one embodiment of the present invention, an absorbent article is provided comprising a primary fastening system and a secondary fastening system. The primary fastening system may comprise at least a first component and at least a second component, the first component interlocking with a respective second component. The secondary fastening system may comprise at least a first secondary fastening member, also referred to herein as a surface fastener. The secondary fastening member may be provided on one of the components of the primary fastening system. The secondary fastening system may be provided with a protective portion on at least a secondary fastening member. The protective portion protects the secondary fastening system to prevent it from accidentally engaging and interfering with fastening of the primary fastening system or becoming contaminated during wearing of the article such that the secondary fastening system is properly configured for use at disposal of the article.

While multiple embodiments are disclosed, still other embodiments of the invention will become apparent to those skilled in the art from the following detailed description, which shows and describes illustrative embodiments of the invention. As will be realized, the invention is capable of modifications in various obvious aspects, all without departing from the spirit and scope of the invention. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter which is regarded as forming the present invention, it is believed that the invention will be better understood from the following description, which is taken in conjunction with the accompanying drawings in which like designations are used to designate substantially identical elements, and in which:

FIG. 1 is a plan view of an absorbent article suitable for use with a secondary fastening system, the absorbent article being in its flat uncontracted state with the body-facing surface facing the viewer.

FIG. 2 is a perspective view of the absorbent article of FIG. 1 in a fastened configuration.

FIG. 3 is a plan view of a slot member of a primary fastening system suitable for use with a secondary fastening system.

FIG. 4 is a plan view of a tab member of a primary fastening system suitable for use with a secondary fastening system.

FIG. 5 is a plan view of interlocking of the slot member of FIG. 3 with the tab member of FIG. 4.

FIG. 6 is an end view of the fastening system shown in FIG. 5.

FIG. 7 illustrates a secondary fastening member comprising a tab extending from a slot member in accordance with one embodiment.

FIG. 8 illustrates a secondary fastening member comprising a tab extending from a tab member in accordance with one embodiment.

FIG. 9 illustrates a secondary fastening member comprising a surface fastener provided on a slot member in accordance with one embodiment.

FIG. 10 illustrates a secondary fastening member comprising a surface fastener provided on a tab member in accordance with one embodiment.

FIG. 11 illustrates a secondary fastening member comprising a tab extending from a slot member, the slot member having a protective portion provided thereon, in accordance with one embodiment.

FIG. 12 illustrates a secondary fastening member comprising a tab extending from a tab member, the tab member having a protective portion provided thereon, in accordance with one embodiment.

FIG. 13 illustrates a diaper having secondary fastening members such as shown in FIG. 8.

FIG. 14 illustrates the diaper of FIG. 13 in a configuration for disposal.

FIG. 15 illustrates a surface fastener with a protective portion provided thereon in accordance with one embodiment.

### DETAILED DESCRIPTION OF THE DRAWINGS

Disposable absorbent articles having a secondary fastening system for fastening the article in a configuration for disposal are provided. Secondary fastening systems suitable for use with disposable absorbent articles are provided.

As used herein, the term “absorbent article” refers to devices that absorb and contain body exudates, and, more specifically, refers to devices that are placed against or in proximity to the body of the wearer to absorb and contain the various exudates discharged from the body. The term “disposable” describes absorbent articles that are not intended to be laundered or otherwise restored or reused (i.e., they are intended to be discarded after a single use and, preferably, to be recycled, composted, or otherwise disposed of in an environmentally compatible manner). A “unitary” absorbent article refers to an absorbent article that is formed of separate parts united together to form a coordinated entity so that it does not require separate manipulative parts such as a separate holder and liner. The term “diaper” refers to an absorbent article generally worn by infants and incontinent persons about the lower torso. It should be understood that the secondary fastening systems taught herein are also applicable to other absorbent articles such as incontinent briefs, incontinent undergarments, diaper holders and liners, feminine hygiene garments, catamenial pads, medical wraps, bibs, bandages, and the like.

“Primary fastening system” refers to a fastening system used to hold the diaper in its in-use configuration about the wearer.

“Secondary fastening system” or “secondary fastening devices” refer to fastening devices used to fasten the diaper in a configuration for disposal and may alternatively be referred to as disposal fastening systems or devices.

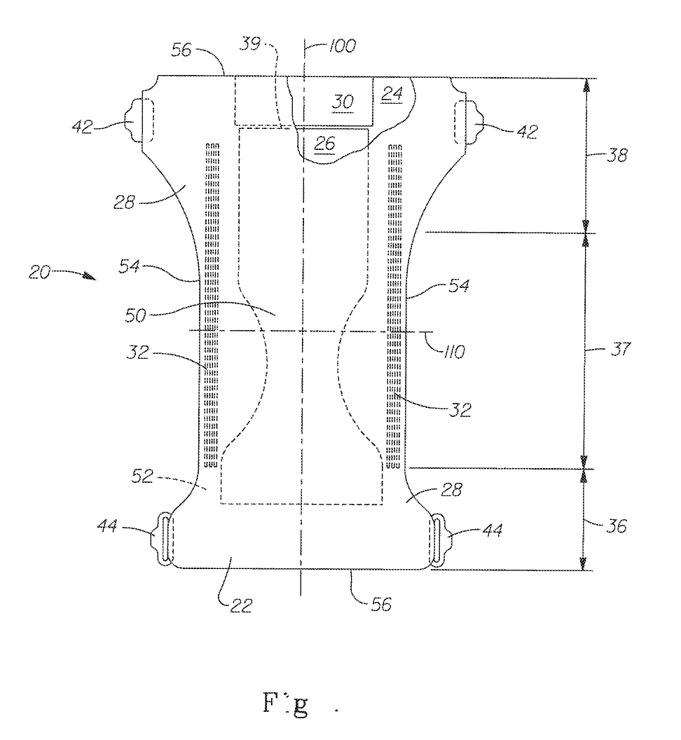

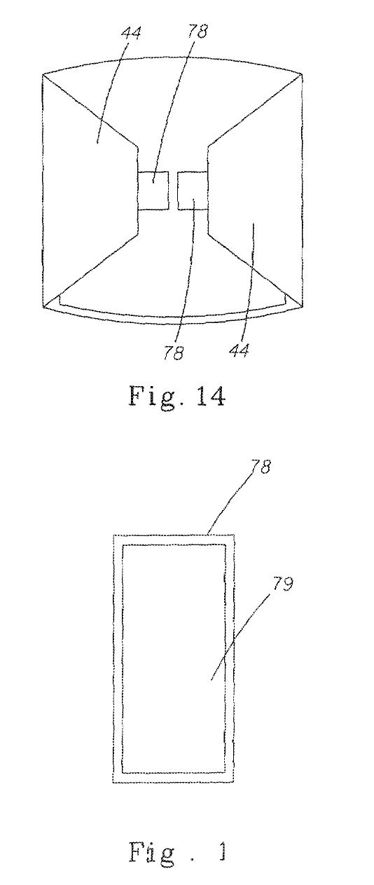

One embodiment of an absorbent article suitable for use with a secondary fastening system as provided herein is the unitary disposable absorbent article, diaper 20, shown in FIG. 1. FIG. 1 does not illustrate a secondary fastening system and reference is made to FIGS. 13 and 14 to illustrate absorbent articles such as that shown in FIG. 1 with secondary fastening systems.

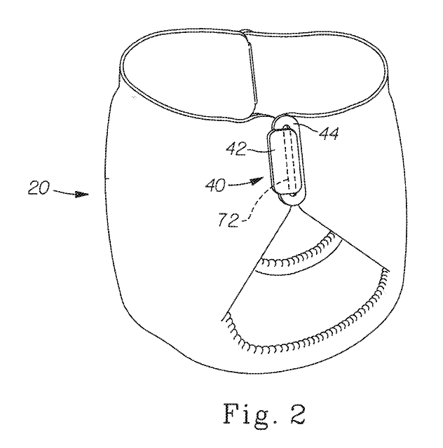

FIG. 1 is a plan view of one embodiment of a diaper 20 with portions of the structure being cut away to more clearly show the construction of the diaper 20. The portion of the diaper 20 that faces or contacts the wearer, the inner surface 50, is oriented toward the viewer. As shown in FIG. 1, the diaper 20 comprises a liquid pervious topsheet 22; a liquid impervious backsheet 24 joined with the topsheet 22; an absorbent core 26 positioned between the topsheet 22 and the backsheet 24; side panels 28; and leg cuffs 32. As used herein, the term “joined” encompasses configurations whereby an element is directly secured to another element by affixing the element directly to the other element, and configurations whereby the element is indirectly secured to the other element by affixing the element to intermediate member(s) which in turn are affixed to the other element. FIG. 2 illustrates the diaper 20 of FIG. 1 in a configuration for wearing.

The diaper 20 is shown in FIG. 1 to have an outer surface 52, an inner surface 50 opposed to the outer surface 52, first waist region 36, a second waist region 38 opposed to the first waist region 36, and a crotch region 37 positioned between the first waist region 36 and the second waist region 38. The diaper 20 also has longitudinal edges 54 and end edges 56. The inner surface 50 of the diaper 20 generally comprises that portion of the diaper 20 that is positioned adjacent to the wearer's body in use (i.e., the inner surface 50 generally is formed by at least a portion of the topsheet 22 and other components joined to the topsheet 22). The outer surface 52 generally comprises that portion of the diaper 20 that is positioned away from the wearer's body (i.e., the outer surface 52 generally is formed by at least a portion of the backsheet 24 and other components joined to the backsheet 24). The diaper 20 also has a longitudinal centerline 100 and a lateral centerline 110.

FIG. 1 shows one embodiment of the diaper 20 in which the topsheet 22 and the backsheet 24 have length and width dimensions generally larger than those of the absorbent core 26. The topsheet 22 and the backsheet 24 extend beyond the edges of the absorbent core 26 to thereby form the periphery of the diaper 20. In alternative embodiments, the length and width dimensions of the topsheet 22, the backsheet 24, and the absorbent core 26 may all be the same, may all be different, or may comprise some combination thereof. While the topsheet 22, the backsheet 24, and the absorbent core 26 may include many different materials and may be assembled in a variety of well-known configurations, example diaper materials and configurations are described generally in U.S. Pat. Nos. 3,860,003, 5,151,092, and 5,221,274.

The absorbent core 26 may be any absorbent means that is generally compressible, conformable, non-irritating to the wearer's skin, and capable of absorbing and retaining liquids such as urine and other body exudates. The absorbent core 26 may be manufactured in any suitable size or shape (e.g., rectangular, hourglass, “T”-shaped, asymmetric, etc.) and from a wide variety of liquid-absorbent materials, including comminuted wood pulp, which is generally referred to as airfelt; creped cellulose wadding; meltblown polymers including coform; chemically stiffened, modified, or cross-linked cellulosic fibers; tissue including tissue wraps and tissue laminates; absorbent foams; absorbent sponges; superabsorbent polymers; absorbent gelling materials; other suitable materials; or combinations of these.

The backsheet 24 may be manufactured from a thin plastic film or a nonwoven web, although other flexible materials may also be used. The backsheet 24, alone or in combination with other materials, prevents the exudates absorbed and contained in the absorbent core 26 from wetting articles that contact the diaper 20. The backsheet 24 may thus comprise a woven or nonwoven material, polymeric films such as thermoplastic films of polyethylene or polypropylene, or composite materials such as a film-coated nonwoven material. The backsheet 24 may be embossed and/or matte finished to provide a more cloth-like appearance. Further, the backsheet 24 may permit vapors to escape from the absorbent core 26 (i.e., breathable) while preventing exudates from passing through the backsheet 24. In one embodiment, the backsheet 26 may comprise a structural elastic-like film (SELF) web. SELF webs suitable for the present invention are more completely described in U.S. Pat. No. 5,518,801.

The topsheet 22 may be compliant, soft feeling, and non-irritating to the wearer's skin. The topsheet 22 is generally liquid pervious, permitting liquids (e.g., urine) to readily penetrate through its thickness. A suitable topsheet may be manufactured from a wide range of materials, such as porous foams; reticulated foams; apertured plastic films; or woven or nonwoven webs of natural fibers (e.g., wood or cotton fibers), other natural fibers, synthetic fibers (including but not limited to polyester, polypropylene, etc.), or a combination of natural and synthetic fibers.

The diaper 20 may comprise leg cuffs 32 to improve containment of liquids and other body exudates. The leg cuffs may be elasticized and may comprise several different embodiments for reducing the leakage of body exudates in the leg regions. The leg cuff may alternatively be referred to as leg bands, side flaps, barrier cuffs, or elastic cuffs. See U.S. Pat. Nos. 3,860,003, 4,909,803, and 4,695,278.

A waist feature 30 may be provided for improving fit and containment. If provided, the waist feature 30 is a portion or zone of the diaper 20 that is intended to elastically expand and contract to dynamically fit the wearer's waist. The elastic waist feature 30 generally extends longitudinally outwardly from at least one of the waist edges 39 of the absorbent core 26 and generally forms at least a portion of the end edge 56 of the diaper 20. Although disposable diapers are generally constructed so as to have two elastic waist features, one positioned in the first waist region 36 and one positioned in the second waist region 38, diapers can be constructed with a single elastic waist feature 30. Further, while the elastic waist feature 30 or any of its constituent elements can include a separate element affixed to the diaper 20, the elastic waist feature 30 may be constructed as an extension of other elements of the diaper 20 such as the backsheet 24, the topsheet 22 or both the backsheet 24 and the topsheet 22. Examples of suitable waist features include those described in U.S. Pat. Nos. 4,515,595, 5,151,092, and 5,221,274.

The diaper 20 comprises a primary fastening system 40 (shown in FIG. 2) that joins at least a portion of the first waist region 36 of the diaper 20 with at least a portion of the second waist region 38 to form leg and waist openings. In one embodiment, the primary fastening system is a macrofastener fastening system comprising first and second interlocking components 42 and 44, described more fully below. The primary fastening system may also work with the waist feature(s) 30 to maintain lateral tension to keep the diaper 20 in place about the wearer. The primary fastening system may be the primary fastening system for joining the first and second waist regions 36 and 38. The primary fastening system generally is strong enough to remain fastened throughout the article's normal time of use when subjected to the dynamic forces created by the wearer's movements. The primary fastening system 40 may be used alone or in conjunction with other fastening means such as hook and loop fasteners, tape fasteners, snaps, buttons, and the like to provide different fastening characteristics. In alternative embodiments, primary fastening systems comprising surface fasteners may be used.

In accordance with some embodiments, a secondary fastening system is provided with the primary fastening system 40 and forms a disposal means for fastening the diaper 20 in a configuration convenient for disposal. The secondary fastening system is not intended to independently hold the diaper 20 in its constructed configuration about the wearer, but rather to complement the primary fastening system by providing additional benefits generally related to disposability. In some embodiments, the secondary fastening system comprises a protected surface fastener provided on one of the first and/or second components of the primary fastening system.

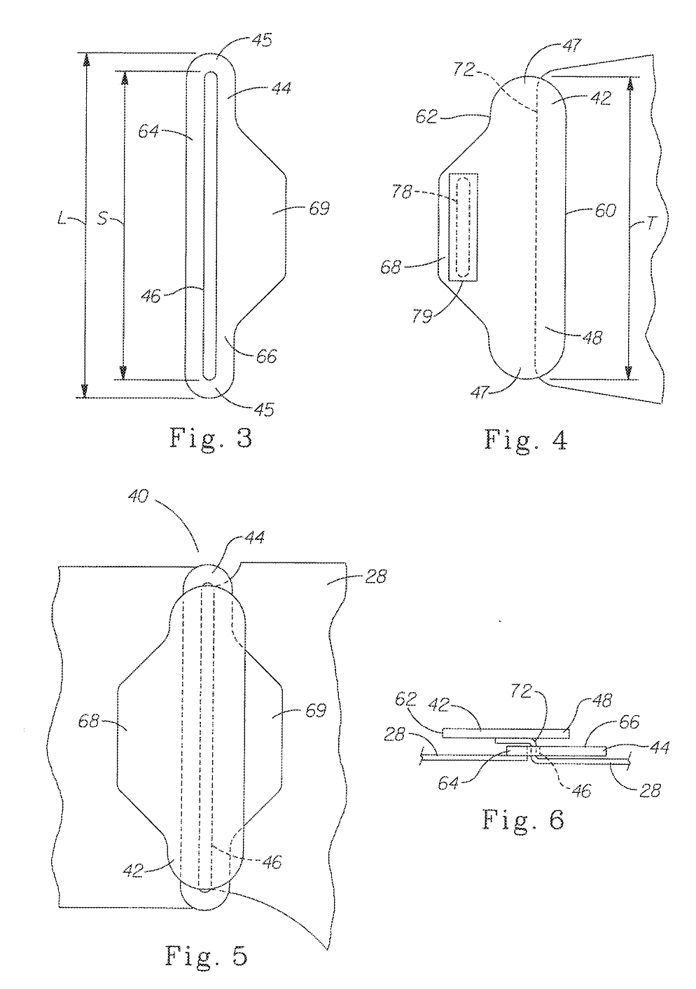

In one embodiment, the primary fastening system comprises a first component that is a tab member or a pair of tab members 42 and a second component that is a slot member or a pair of slot members 44. FIG. 3 illustrates a slot member 44 in more detail, FIG. 4 illustrates a tab member 42 in more detail, and FIG. 5 illustrates the interlocking of the tab member 42 and the slot member 44. As shown in FIG. 4, the tab member 42 includes a generally elongate member having a proximal edge 60, a distal edge 62, a lip portion 48, and a length T. As shown in FIG. 3, the slot member 44 includes an inboard portion 64, an outboard portion 66, and a slot 46 disposed between the inboard portion 64 and the outboard portion 66. The slot member 44 has a length L and the slot 46 has a length S. The slot member 44 may also include a grip portion 69 like that of the tab member 42. Grip portions may be provided on the tab member 42 and the slot member 44. The grip portions 68 and 69 help the user grip the tab member 42 and slot member 44 respectively when fastening or releasing the primary fastening system.

In a simple form, the primary fastening systems are fastened by passing the tab member 42 through the slot 46 of the slot member 44. In embodiments including two or more slot members 44 that engage one another, one of the slot members 44 is passed through the other slot member 44 to fasten the device. Once the tab member 42 has been passed through the slot member 44, as shown in FIG. 6, the lip portion 48 of the tab member 42 is rotated into a plane generally parallel with the plane of the slot member 44 such that at least a part of the lip portion 48 is overlapping at least a part of the outboard portion 66 of the slot member 44. In this configuration, the lip portion 48 of the tab member 42 will prevent the tab member 42 from slipping back through the slot 46 and disengaging the fastening system. A portion of the tab member 42 or the material of the article to which the tab member 42 is joined will extend into the slot 46, as shown in FIG. 6. The material in the slot 46 acts to resist forces in shear which tend to direct the tab member 42 and the slot member 44 apart.

The tab member 42 may be of any size and/or shape and may be made from any suitable material. The tab member 42 may be sized to fit through the slot 46 of the slot member 44 with little or no bending or deformation of either component. The shape of the tab member 42 may be based on the end use of the fastening system, and may be one or more of aesthetically pleasing, easy to hold and maneuver, and capable of maintaining the device in a fastened configuration throughout the intended period of use when subjected to expected forces and external conditions. The tab member 42 is coupled to a retaining material at a line of attachment 72. The tab member 42 may be designed so that the member or a portion or portions of the tab member 42 are located outside of a “compressive” region of a wearer. A compressive region of the wearer is a region in which the natural bodily movement of the wearer will move towards the region. An “expanding” region of a wearer, however, is a region in which the natural body movement of the wearer will move away from the region. There are several compressive and expanding regions on the body of a wearer, especially where there are joints that bend such as knees, elbows, shoulders, hips, ankles, wrists, etc. To determine whether a particular region of the body is a compressive or expanding zone, the change in the surrounding anatomical structure of the region is examined as a joint is moved from a “neutral” position to a bent position. A neutral position is when the joint is not bent, i.e., the position with the muscles generally relaxed and not attempting to flex the joint. If the surrounding anatomical structure, e.g., tissues, bones, etc., would tend to compress or bend an object on the body's surface as the joint is moved from a neutral to a flexed position, the region is a compressive region. If the surrounding anatomical structure would tend to expand or stretch an object on the body's surface, the region is an expanding region. The principle of avoiding compressive regions and utilizing expanding regions may also be applied to the design of tab members for articles other than diapers such as, but not limited to, sanitary napkins, bibs and wraps placed on other parts of the wearer.

The tab member 42 may include one or more tab elements for insertion into the slot 46 of the slot member 44. A tab member 42 with a single tab element is shown in FIGS. 1, 2, and 5. However, embodiments are contemplated where the tab member 42 comprises more than one tab element. If the tab member 42 includes more than one tab element, the tab elements may be operatively associated with each other. The term “operatively associated” refers to elements that are directly or indirectly joined together so as to function generally as a single element. The term “directly joined” refers to elements that are joined to each other without any intermediate elements joined therebetween, except for the means joining the elements (i.e., the adhesive). The term “indirectly joined” refers to elements joined with each other by means of an element or elements other than the joining means. The tab member 42, regardless of the number of tab elements, may function as a single element of the fastening system. This reduces the complexity of the fastening system and ensures that a single fastening motion can engage the entire tab member 42, whether the tab member 42 includes one or more tab elements.

The tab member 42 may be unitary with the article to which it is attached, or it may be a separate element joined thereto. The tab member 42 may be joined to the article at any location. In a disposable absorbent article embodiment, such as that shown in FIG. 1, the tab member 42 may be an extension of the material making up the side panel 28. In such cases, it may be preferable to provide additional material or to process the material of the side panel 28 so as to change some of its physical properties. For example, it may be desirable that the side panel 28 be extensible and the tab member 42 not be extensible. Further, it may be desirable to aid in proper fastening that the tab member 42 be stronger and/or stiffer than the side panel 28. Alternatively, the tab member 42 may be a separate element that is joined to the article. In such cases, the tab member 42 may be made of the same or different materials than the article to which it is attached, making it easy to match the exact properties of the fastening system to the intended use. Further, the material from which the tab member 42 is made can be reinforced and/or weakened at certain locations to help provide the desired flexibility and stiffness to the fastening system. In one embodiment the tab member 42 may be reinforced and/or weakened at one or both of its longitudinal ends 47. In other embodiments, the grip tab 69 of the tab member 42 may be reinforced and/or weakened. Methods of weakening the material include scoring, cutting, thinning, bending, heat treating, chemical treating, and the like. Methods of reinforcing include heat or chemical treating the material, adding material, increasing the thickness, etc.

The materials that make up the tab member 42 may be chosen based on the end use of the fastening system. For example, if the fastening system is to be used in a diaper, as shown in FIG. 1, the tab member 42 may include any material suitable for such a use, including but not limited to plastics, films, foams, nonwoven webs, woven webs, paper, laminates, steel, fiber reinforced plastics, etc., or combinations thereof. In embodiments where the fastening system is used near or against the skin of a human or animal, the materials making up the tab member 42 may be flexible. The flexibility allows the fastening system to conform to the shape of the body and thus, reduces the likelihood that the fastening system will irritate or injure the wearer's skin.

The slot member 44 is that portion of the primary fastening system through which the tab member 42 is passed in order to engage or fasten the device. The length S of the slot 46 is generally greater than or equal to the length T of the tab member 42 such that the tab member 42 is easily passed through the slot 46 without undue bending or deformation of either component. The slot may alternatively include a slit, which is defined as a slot having essentially no gap other than that left by a cutting process.

The slot member 44 may be of any size and/or shape and may be made from any suitable material. The shape of the slot member 44 and the materials that make up the slot member 44 may be based on the end use of the fastening system. For example, in end uses such as diapers, the slot member 44 generally is designed to be skin friendly, i.e., not harmful to the wearer's skin. Thus, it may be desirable to round the edges of the fastening system and to size the slot(s) 46 so as to minimize the likelihood that skin will be caught in the device. One way of doing this is to work the edges of the slot 46 such that they are not sharp. Another way is to make the fastening system more skin friendly includes minimizing the thickness of the slot member 44 (e.g., less than about 0.05 inches) or to design the tab member 42 or slot member such that the slot 46 is filled in when the fastening system is closed. Yet another way is to provide a soft or compressible material on at least the surface of the fastening system which faces the wearer. A further way to make the fastening system more skin friendly, as described above with reference to the tab member 42, is to design the shape of the slot member 44 so that the slot member or a portion or portions of the slot member 44, when fastened, may be located in an expanding region of a wearer so that the wearer's body will not press up against the slot member 44 as the wearer moves or to minimize the portion of the slot member 44 that is located in a compressive region as described above with respect to the tab member 142.

The slot member 44 may be made of materials the same as or different from the tab member 42, including plastics, films, foams, nonwoven webs, woven webs, paper, laminates, steel, fiber reinforced plastics, other suitable materials, or combinations thereof. As with the tab member 42, the materials making up the slot member 44 may be flexible. The slot member 44 may be stiff enough in the lateral direction so as not to deform and let the tab member 42 disengage under normal in use fastening forces. The material from which the slot member 44 is made can be reinforced or weakened at certain locations to help provide the desired flexibility and stiffness to the fastening system. In one embodiment, the slot member 44 may be reinforced and/or weakened at one or both of its longitudinal ends 45. In other embodiments, the grip tab 69 of the slot member 44 may be reinforced or weakened. Methods of weakening the material include scoring, cutting, thinning, bending, heat treating, chemical treating, and the like. Methods of reinforcing include heat or chemically treating the material, adding material, increasing the thickness, and the like.

The slot member 44 may be unitary with the article to which it is attached, or it may be a separate element joined thereto. Further, the slot member 44 may be joined to the article at any suitable location. In a disposable absorbent article embodiment such as a diaper 20, the slot member 44 may be an extension of the material making up the side panel 28 or any other portion of the diaper 20. In some embodiments a tab member 42 and a slot member 44 can be provided at a first waist region and a tab member 42 and a slot member 44 can be provided at a second waist region. Alternatively, the slot member 44 may include the material that makes up the outer surface 52 of the diaper 20. Alternative embodiments include the slot(s) 46 of the slot member 44 being cut through any number of layers of the article to which the fastening system is attached, such as both the topsheet 24 and the backsheet 26. Alternatively, the slot member 44 may be a separate element that is joined to the article. The slot member 44 may be joined to the article in a fixed position, or it may be adjustably joined to the underlying article. In any such case, the slot member 44 may be made of the same or different materials than the article to which it is attached. Reference is made to U.S. Pat. No. 6,936,039 for specifics on the tab member 42 and slot member 44 of the fastening system. Any suitable interlocking fasteners may be used in place of the tab and slot interlocking primary fastening system. Alternative exemplary suitable interlocking fasteners include in plane engaging fastening members. Additional suitable fasteners include those described in U.S. Pat. Nos. 6,432,098; 6,251,097; and U.S. Patent Application Nos. 2003/0233082; 2007/0074381; 2007/0093769; 2007/0078427; 2007/0078426.

The secondary fastening system of the present invention may include a secondary fastening member provided on the diaper 20. The secondary fastening member may be provided on a portion of the primary fastening system or member. In the embodiment of FIG. 4, a tab member 42 of the primary fastening system has a secondary fastening member 78 provided thereon. Secondary fastening member 78 is provided with a protective portion 79. Generally, the secondary fastening member 78 may be used to fasten the diaper 20 in a configuration for disposal, and protective portion 79 covers secondary fastening member 78 prior to the diaper 20 being configured for disposal. Thus, in one embodiment, the tab member 42 may include secondary fastening member 78 located adjacent the distal edge 62 of the tab member 42 or adjacent the grip portion 68, as shown in FIG. 4. In alternative embodiments, the secondary fastening member 78 may be provided in other configurations and/or may be located elsewhere on the tab member 42. The secondary fastening member may be used to provide the fastening system with the ability to better resist shear or peel forces, greater adjustability, or other properties.

Alternatively or additionally, a secondary fastening member 78 may be provided on the slot member 44. For example, the slot member 44 may include secondary fastening member 78 located adjacent the inboard portion 64, the outboard portion 66, the grip portion 68, or any other portion of the slot member 44. The secondary fastening member may be used to fasten the diaper 20 in a configuration for disposal. As noted with regard to the tab member 42, the secondary fastening member can be used to provide the fastening system with the ability to better resist shear or peel forces, greater adjustability, a disposal feature, and/or other features.

In accordance with one embodiment of the present invention, a secondary fastening member 78 may be provided on each component of a pair of components of the primary fastening system. Thus, for example, a secondary fastening member 78 may be provided on each of two slot members 44, or on each of two tab members 42. In various embodiments, a secondary fastening member 78 on the slot member 44 may function together with a secondary fastening member disposed on the tab member 42. Alternately, a secondary fastening member 78 on either of the tab member 42 or the slot member 44 may function independently, or may function with another portion of the diaper or absorbent article. The secondary fastening member 78, whether disposed on the tab member 42, on the slot member 44, or both, may comprise a surface fastener, or may comprise a macrofastener, each described more fully below.

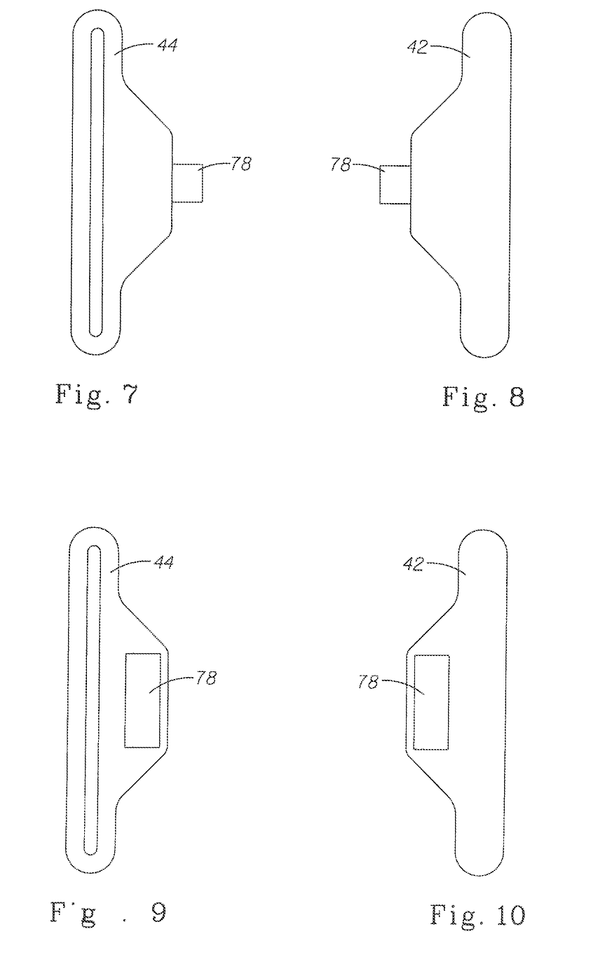

FIGS. 7-15 illustrate further embodiments wherein the secondary fastening member 78 comprises a surface fastener. Such surface fastener may comprise hooks, loops, adhesive, cohesive, hermaphroditic fasteners, magnetic material, static electricity, or any combination of these or other surface fasteners. In general, surface fasteners comprise engaging elements on a surface of a component, wherein said engaging elements are configured to form at least temporary bonds in a face-to-face (i.e., surface-to-surface) relationship with receiving elements on another surface.

In the embodiments of FIGS. 7-15, tab members and slot members such as shown in FIGS. 3-5 are shown with the secondary fastening system.

Returning to FIGS. 7-15, wherein the secondary fastening member 78 comprises a surface fastener, the surface fastener is protected prior to using the secondary fastening member 78. Thus, for example, where the secondary fastening member 78 comprises an adhesive or a cohesive, a protective portion 79 may be provided over the adhesive or cohesive and removed when it is desired to use the secondary fastening member 78. Where the secondary fastening member 78 comprises hooks of a hook-and-loop closure, the hooks are protected by protective portion 79 such that they cannot engage until desired. Such protection may be in the form of a covering, may comprise the hooks being folded down or adhered until exposed, may comprise the secondary fastening member 78 being folded in on itself, or other acceptable means. Regardless of the type of surface fasteners on the secondary fastening member 78, the protective portion covers the surface fasteners' engaging elements to prevent unintended engagement of them until such time the user desires to use them.

Protecting the surface fastener is of utility when the surface fastener is provided on a component of a macrofastener primary fastening system. In an embodiment wherein an adhesive secondary fastening member 78 is provided on one of, for example, a tab member 42 or a slot member 44, failure to protect the adhesive secondary fastening member 78 may lead to difficulties in fitting of the diaper to the wearer, wearing of the diaper by the wearer, and use of the adhesive secondary fastening member 78. In fitting the diaper to the wearer, an adhesive on either of the tab member 42 or the slot member 44 may engage the other component of the primary fastening system and thus need to be disengaged. This may be an inconvenience to the caregiver, and may lead to contamination-induced performance problems. If the secondary fastening system becomes contaminated prior to disposal, it may not function as desired to keep the article properly configured for disposal. During wear of the diaper, the adhesive secondary fastening member 78, depending on placement thereof, may engage the diaper, clothing worn over the diaper, or other. Again, this may be an inconvenience to the caregiver and may lead to contamination-induced performance problems, thus rendering the secondary fastening system ineffective for disposal. Similarly, in an embodiment wherein a hook-type secondary fastening member 78 is provided on one of, for example, a tab member 42 or a slot member 44, failure to protect the hook type secondary fastening member 78 may lead to problems in fitting of the diaper to the wearer and wearing of the diaper by the wearer. In fitting the diaper to the wearer, a hook type fastener on either of the tab member 42 or the slot member 44 may become entangled with the other component of the primary fastening system or the diaper and thus need to be disengaged. During wear of the diaper, the hook type secondary fastening member 78, depending on placement thereof, may engage the diaper, clothing worn over the diaper, or other.

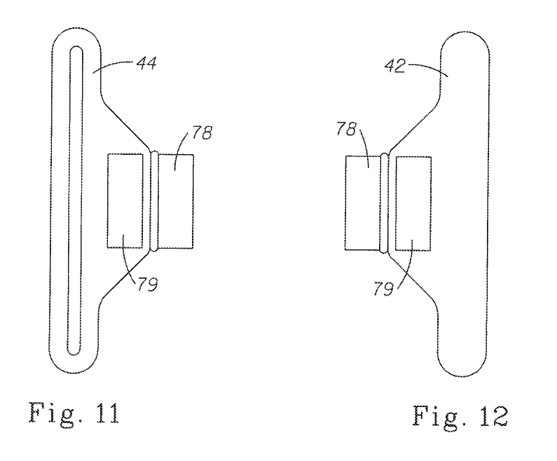

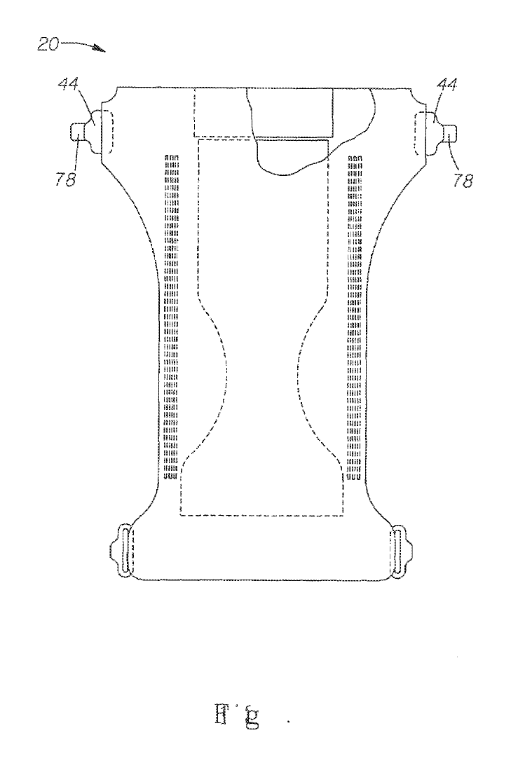

FIGS. 7, 9, and 11 illustrate secondary fastening members 78 comprising a surface fastener provided on a slot member 44. FIG. 13 illustrates a diaper having secondary fastening member surface fasteners provided on each of a pair of slot members of the primary fastening system thereof. FIG. 14 illustrates the diaper of FIG. 13 in a configuration for disposal. FIGS. 8, 11, and 13 illustrate secondary fastening members 78 comprising a surface fastener provided on each of a pair of tab member 42 of the primary fastening system. In one embodiment, the fastening members 78 of FIGS. 7 and 8 may be similar, the fastening members 78 of FIGS. 9 and 10 may be similar, and the fastening members of FIGS. 11 and 12 may be similar, though in alternative embodiments they may be different. A protective portion 79 is provided with the secondary fastening member 78. As shown in FIG. 15, the protective portion 79 may comprise a layer over the secondary fastening member 78, such as a peel-away strip provided over an adhesive or cohesive, or may be provided by the configuration of the secondary fastening member 78, a loop layer provided over hooks of a hook-and-loop secondary fastening system, a substrate (such as a nonwoven, a film, a laminate, or the like) provided over hooks or loops of a hook-and-loop secondary fastening system, or other. Any substrate which covers the engaging elements of the secondary fastening member and prevents them from engaging until such time the user desires to use the secondary fastening member, yet readily releases from the secondary fastening member at that time can be a suitable substrate. This substrate may be a separate piece or element added to cover the engaging elements. Alternatively, the protective portion may be provided by the configuration of the surface fastener. For example, hooks may be folded down or adhered until exposed, or part or all of the secondary fastening member 78 may be folded over onto a protective portion or onto itself. For adhesive or cohesive secondary fastening members 78, the protective portion 79 may further include a surface designed to release that is in face-to-face contact with the adhesive engaging elements such that the protective portion readily releases from the adhesive engaging elements when the user removes the protective portion. Release layers of varying forms and chemistries are known in the art.

FIGS. 7 and 8 illustrate embodiments of a secondary fastening member 78 comprising a tab extending from the primary fastening system. FIG. 7 illustrates the tab extending from a slot member 44. FIG. 8 illustrates the tab extending from a tab member 42. The tab may comprise a surface with an adhesive thereon, a hook or loop of a hook-and-loop closure, or other surface fastener as previously described. As previously described, a protective portion may be provided with the tab. The tab may be a discrete, separate element added to the primary fastening member or may be formed from an extension of any layer of a primary fastening member. Further, the protective portion may be a discrete, separate element added to the primary fastening member or may be formed from an extension of any layer of a primary fastening member.

FIGS. 9 and 10 illustrate embodiments of a secondary fastening member 78 comprising a surface fastener provided on the primary fastening system. FIG. 9 illustrates the surface fastener on a slot member 44. FIG. 10 illustrates the surface fastener on a tab member 42. The surface fastener may comprise an adhesive, a hook or loop of a hook-and-loop closure, or other surface fastener as previously described. The surface fastener may be provided on any suitable location of the primary fastening system. In the embodiments shown, the surface fastener is provided generally on a handling portion of the primary fastening system. As previously described, a protective portion is provided with the surface fastener.

FIGS. 11 and 12 illustrate embodiments of a secondary fastening member 78 comprising a tab extending from the primary fastening system with the protective portion 79 being provided on the primary fastening system. FIG. 11 illustrates the secondary fastening member 78 on a slot member 44. FIG. 12 illustrates the secondary fastening member 78 on a tab member 42. The tab may comprise a surface with an adhesive thereon, a hook or loop of a hook-and-loop closure, or other surface fastener as previously described. The protective portion 79 is provided on the primary fastening system at a location suitable for receiving the secondary fastening member. The secondary fastening member 78 may be folded toward the protective portion 79 and engaged with the protective portion to protect the secondary fastening member 78 until use. For use, the secondary fastening system may be pulled away from the protective portion 79. Thus, for example, where the secondary fastening system comprises hooks, the protective portion may comprise loops; where the primary fastening system comprises an adhesive, the protective portion may comprise a releasable protective strip; etc.

FIG. 13 illustrates a diaper 20 with secondary fastening system surface fasteners 78 such as described with FIG. 7 extending from the tab members 44 of the primary fastening system. FIG. 14 illustrates the diaper 20 of FIG. 13 in a configuration for disposal wherein the secondary fastening systems are engaged to fasten the diaper in such configuration.

The secondary fastening member 78 may be placed at any suitable location on the primary fastening system. Preferably, the secondary fastening member 78 is adjacent the distal end of the primary fastening system. The secondary fastening member 78 may also be located on the entire surface or adjacent a proximal end of the primary fastening system.

During fitting of the diaper to the wearer, the secondary fastening member 78 may be protected so as not to engage the other component of the primary fastening system or any other portion of the diaper. Similarly, during wearing of the diaper by the wearer, the secondary fastening member 78 may be protected so as not to engage portions of the diaper or clothing worn over the diaper. After the diaper is removed, the secondary fastening member 78 may be prepared for use, if needed. This may comprise removing a layer such as a peel away strip or a loop layer, exposing an adhesive, hooks, or other. After the diaper is removed and the secondary fastening member 78 is prepared, the diaper is manipulated into a configuration for disposal and the secondary fastening member 78 is engaged to fasten the diaper in such configuration. Manipulating the diaper into a configuration for disposal may comprise rolling the diaper. For example, the caregiver may remove the diaper by pulling down the front of the diaper, and may use the front of the diaper to wipe away any lingering excrement. The caregiver then may proceed to roll the front of the diaper to the back of the diaper, thus folding the excrement into the diaper. After the diaper has been rolled, the secondary fastening member 78 may be used to fasten the diaper in the rolled configuration. In accordance with this example, the secondary fastening member 78 may be provided on a component of the primary fastening system located on the rear of the diaper. In alternative embodiments, the secondary fastening member 78 may be provided in a location suitable for back to front rolling. In such an embodiment, the secondary fastening member may be placed on a component of the primary fastening member located on the front of the diaper. In some embodiments, only one secondary fastening member 78 is provided. In others, at least two secondary fastening members 78 are provided. When more than one secondary fastening members 78 are provided, many configurations are possible. For example, when more than one secondary fastening member is provided, each secondary fastening member 78 may be located on primary fastening member components on opposing sides at the rear of the product, each secondary fastening member 78 may be located on primary fastening member components on opposing sides at the front of the product, one may be located on the front and one of the rear of the article on either the same or opposing sides of the article, or secondary fastening members may be located on each corner of the diaper. For example, a secondary fastening member 78 may be provided on each of the tab members 42 and the slot members 44 such that the diaper may configured in any suitable manner for disposal and fastened in such configuration.

The secondary fastening member 78 may be configured to engage with any portion of the article to secure the article for disposal. For example, when the secondary fastening member includes adhesive fastening elements, the adhesive fastening elements can be readily configured to join securely to many existing diaper components, such as a backsheet or any other portion such as any outer surface of the diaper. Other surface fasteners suitable for use as engaging elements of a secondary fastening member may require more specialized materials to mate with to form a suitably secure fastening for disposal. For example, cohesive or hook or loop engaging elements as secondary fastening member 78 may require a dedicated, separate material to provide a suitably secure fastening for disposal. Alternatively, an existing material (such as a backsheet or other outer surface of a diaper) can be altered to engage with the secondary fastening elements. With specific reference to a secondary fastening member 78 comprising hooks or loops, complementary loops or hooks, respectively, may be provided on a portion of the diaper to be engaged when configuring the diaper for disposal. All or part of the material of the outer surface 52 of the diaper 20 may include loops or hooks for engaging the hooks or loops of the secondary fastening member 78. Preferably, the engaging elements are positioned such that once the protective portion is removed, the engaging elements face the body facing surface of the absorbent article for easiest disposal.

In alternative embodiments, the fastening system may include a tab member 42 and a slot member 44 located on opposite sides of the diaper 20 in the same waist region. This configuration provides one means for using the tab member 42 and the slot member 44 to fasten the diaper 20 in a disposal configuration. In such embodiments, either waist region can be folded inwardly and rolled toward the other waist region. The side panels 28 can then be wrapped around the folded and rolled portion of the diaper 20. The tab member 42 and the slot member 44 located in the same waist region can then be engaged to hold the diaper in a disposal configuration. Another alternative embodiment of the diaper 20 includes a fastening system having two slot members 44 that are engageable. Other embodiments may include one or more tab members 42 having slots 46 so as to allow for alternative attachment and disposal. If the slot member 44 includes a grip tab 69, the interlocked slots can be further secured by passing the grip tab 69 through the slot 46 of the opposing slot member 44.

Protective portion 79 may cover the entire surface area of the engaging elements of the secondary fastening member or a portion of the surface area of the engaging elements of the secondary fastening member. For example, the protective portion 79 may cover at least about 25% of the area of the engaging elements of the surface fastening member, or alternatively the protective portion 79 may cover at least about 50%, 75% or 100% of the area of the engaging elements of the surface fastening member.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention. The use of a fastener of the present invention, for example, may include a fastener for a belt-type absorbent article in which the belt wraps around the wearer and connects one waist region to itself, a fastener that connects one waist region to the other waist region, a disposal fastening system, or any combination thereof. A belt-type fastener may comprise a separate belt that may be joined to the article through a fastener of the present invention, slits, loops, slots, buckles, ties, or any other suitable fastener. Alternatively, a belt can be joined to a portion of the article such as the front or rear waist region and may connect that portion of the article to the wearer and/or to another portion of the article through a fastener of the present invention, slits, loops, slots, buckles, ties, or any fastener known in the art. The fastener of the present invention may also include either a primary fastener, a secondary fastener, or both in a fastening system such as the one disclosed in U.S. Pat. No. 4,699,622. In addition, a fastener of the present invention may also be used in an absorbent article that has separable components in order to hold or attach the separable component(s) to some part of the assembly. The separable component(s) may be, for example, an absorbent core and a liner, or a set of suspenders or other garment from which the absorbent article is supported. In addition, the present invention may be directed to sanitary napkin including a primary fastening system and a secondary fastening system, a bib including a primary fastening system and a secondary fastening system, or a wrap including a primary fastening system and a secondary fastening system. It should also be clear that the fasteners of the present invention may be readily adapted to any of the product forms described above. The present invention is therefore intended to cover in the appended claims all such changes and modifications that are within the scope of this invention.

For any of the embodiments shown herein, the primary fastening system may include any known interlocking macrofasteners, including tabs and slots, rods and sockets, buttons, zippers, snaps, arrows and slots, and the like. The secondary fastening system may include any surface fasteners having a protective portion covering at least a portion of the engaging elements of the surface fasteners.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A fastening system comprising a primary fastening system and a secondary fastening system,
wherein said primary fastening system comprises at least a first component and at least a second component, wherein said first component interlocks with said second component;
wherein said secondary fastening system is located on at least a portion of said primary fastening system;
wherein said secondary fastening system comprises a secondary fastening member having a first surface and a protective portion configured to cover at least a portion of said first surface.

2. The fastening system of claim 1, w herein said secondary fastening member has engaging elements on said first surface.

3. The fastening system of claim 1, wherein said protective portion covers at least about 50% of the surface area of said first surface.

4. The fastening system of claim 1, wherein said protective portion covers at least about 75% of the surface area of said first surface.

5. The fastening system of claim 1, wherein said protective portion covers about 100% of the surface area of said first surface.

6. The fastening system of claim 1, wherein said primary fastening system includes interlocking fasteners selected from the group consisting of tab and slot, rod and socket, arrow and slot, button and button hole, in-plane engaging fastener, and any combination thereof.

7. The fastening system of claim 2, wherein said engaging elements include surface fasteners selected from the group consisting of adhesive, cohesive, hook, loop, hermaphrodidic, magnetic, static, and any combination thereof.

8. The fastening system of claim 1, wherein said primary fastening system is configured to secure an article to a wearer and said secondary fastening system is configured to secure a used article in a configuration for disposal.

9. The fastening system of claim 8, wherein said article is selected from the group consisting of a disposable article, an absorbent article, a diaper, a sanitary napkin, a bib, and a wrap.

10. An article having a primary fastening system for fastening said article in a configuration for wearing, said primary fastening system comprising at least a first component and at least a second component, wherein said first component interlocks with said second component; and a secondary fastening system located on at least a portion of said primary fastening system, said secondary fastening system comprising a secondary fastening member having a first surface and a protective portion configured to cover at least a portion of said first surface.

11. The fastening system of claim 10, wherein said primary fastening system includes interlocking fasteners selected from the group consisting of tab and slot, rod and socket, arrow and slot, button and button hole, in-plane engaging fastener, and any combination thereof.

12. The fastening system of claim 10, wherein said secondary fastening member has engaging elements on said first surface, wherein said engaging elements include surface fasteners selected from the group consisting of adhesive, cohesive, hook, loop, hermaphrodidic, magnetic, static, and any combination thereof.

13. A fastening system comprising a primary fastening system and a secondary fastening system,
wherein said primary fastening system comprises at least a first component and at least a second component;
wherein said secondary fastening system is located on at least a portion of said primary fastening system;
wherein said secondary fastening system comprises a secondary fastening member having a first surface and a protective portion configured to cover at least a portion of said first surface;
wherein said secondary fastening member is located adjacent to the distal edge of said primary fastening system.

14. The fastening system of claim 13, wherein said primary fastening system includes interlocking fasteners selected from the group consisting of tab and slot, rod and socket, arrow and slot, button and button hole, in-plane engaging fastener, and any combination thereof.

15. The fastening system of claim 13, wherein said secondary fastening member has engaging elements on said first surface, wherein said engaging elements include surface fasteners selected from the group consisting of adhesive, cohesive, hook, loop, hermaphrodidic, magnetic, static, and any combination thereof.

16. The fastening system of claim 13, wherein said primary fastening system is configured to secure an article to a wearer and said secondary fastening system is configured to secure a used article in a configuration for disposal.

17. The fastening system of claim 16, wherein said article is selected from the group consisting of a disposable article, an absorbent article, a diaper, a sanitary napkin, a bib, and a wrap.
